(19) United States
(12) Patent Application Publication
(10) Pub. No.: US 2023/0293325 A1
(43) Pub. Date: Sep. 21, 2023

(54) NON-FORSHORTENING BALLOON EXPANDABLE STENT FRAME

(71) Applicant: Merit Medical Systems, Inc., South Jordan, UT (US)

(72) Inventor: Christopher Cindrich, Highland, UT (US)

(21) Appl. No.: 18/183,614

(22) Filed: Mar. 14, 2023

Related U.S. Application Data
(60) Provisional application No. 63/269,387, filed on Mar. 15, 2022.

(51) Int. Cl. A61F 2/91

(52) U.S. Cl. CPC A61F 2/91

(57) ABSTRACT

Stent devices deployable at a treatment site within a lumen of a patient's body are disclosed. The stent devices include a plurality of discrete stent rings coupled to a coupling member. The discrete stent ring includes a plurality of expandable structures. The expandable structure includes a longitudinal strut and a plurality of strut arms extendable from the longitudinal strut. The coupling member is a polymeric tube or a plurality of elongate filaments. The longitudinal struts are coupled to the coupling member.

### RELATED APPLICATIONS

This application claims priority to U.S. Provisional Application No. 63/269,387, filed on Mar. 15, 2022 and titled, “Non-Foreshortening Balloon Expandable Stent Frame,” which is hereby incorporated by reference in its entirety.

### TECHNICAL FIELD

The present disclosure relates generally to devices to treat organs containing a calculus. More specifically, the present disclosure relates to balloon expandable stent devices deployable within a lumen of a patient's body. In some embodiments, the present disclosure relates to non-foreshortening balloon expandable stent devices.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a stent device.

FIG. 2 is a perspective view of an embodiment of a stent frame of the stent device of FIG. 1.

FIG. 3A is a side view of the stent frame of FIG. 2 in a radially compressed state.

FIG. 3B is a side view of the stent frame of FIG. 2 in a radially expanded state.

FIG. 4A is a side view of the stent device of FIG. 1 in a radially compressed state.

FIG. 4B is a side view of the stent device of FIG. 1 in a partial radially expanded state.

FIG. 4C is a side view of the stent device of FIG. 1 in a radially expanded state.

FIG. 5A is a side view of the stent device of FIG. 1 disposed in a vessel adjacent a treatment site in the radially compressed state.

FIG. 5B is a side view of the stent device of FIG. 1 disposed in a vessel adjacent a treatment site in the radially expanded state.

FIG. 6 is a perspective view of another embodiment of a stent device.

### DETAILED DESCRIPTION

In certain instances, stents may be deployed in various body lumens for a variety of purposes. Stents may be deployed, for example, in the vascular system for a variety of therapeutic purposes, including the treatment of occlusions within the lumens of that system. The current disclosure may be applicable to stents designed for the central venous (“CV”) system, peripheral vascular (“PV”) stents, abdominal aortic aneurysm (“AAA”) stents, bronchial stents, esophageal stents, biliary stents, coronary stents, gastrointestinal stents, neuro stents, thoracic aortic endographs, or any other stent or stent graft.

In some instances, stents foreshorten (e.g., shorten in length) when radially expanded during deployment. Foreshortening results in a stent length shorter than a stent length prior to radial expansion. Foreshortening of the stent can cause inaccurate positioning of the stent relative to a treatment site, resulting in incomplete treatment of the treatment site. For example, the stent may be deployed at a stricture to expand the stricture. A stricture designated to treatment has a longitudinal length or the vessel has a length along which treatment is desired. In some instances, a length of the stent may be equivalent or longer than the length of the stricture when radially compressed but may be shorter than the length of the stricture when radially expanding, resulting in incomplete treatment of the stricture.

As used herein, the term “stent device” refers to a prosthesis configured for use within bodily structures, such as within body lumens. The stent device may comprise a plurality of discrete stent frames and a coupling member. The stent frames may comprise a plurality of expandable structures coupled together. Each expandable structure can include a longitudinal strut and strut arms extending therefrom. A length of the stent frame can be equivalent in both a radially compressed state and an expanded state. In other words, the stent frame may be non-foreshorten when radially expanded. In certain embodiments, the stent device may be balloon expandable, meaning the stent device is radially expanded by an expandable balloon from the radially compressed state to the expanded state during deployment. In other embodiments, the stent device may be self-expanding, meaning that the stent device is capable of expanding from the radially compressed state to the expanded state without application of an external force. In some embodiments, the plurality of stent rings are coupled to a coupling member. The coupling member can be a polymeric tube or a plurality of filaments.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

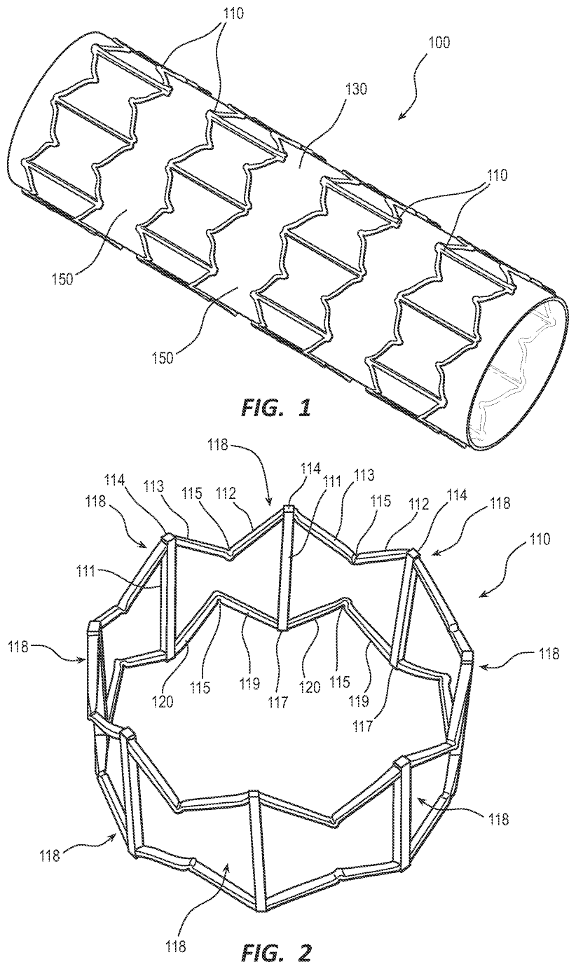

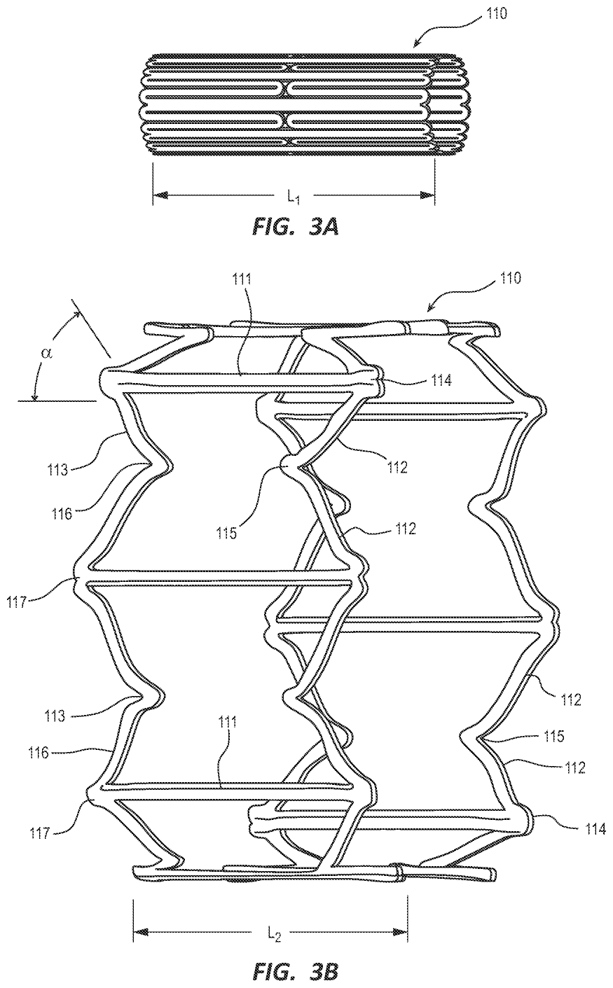

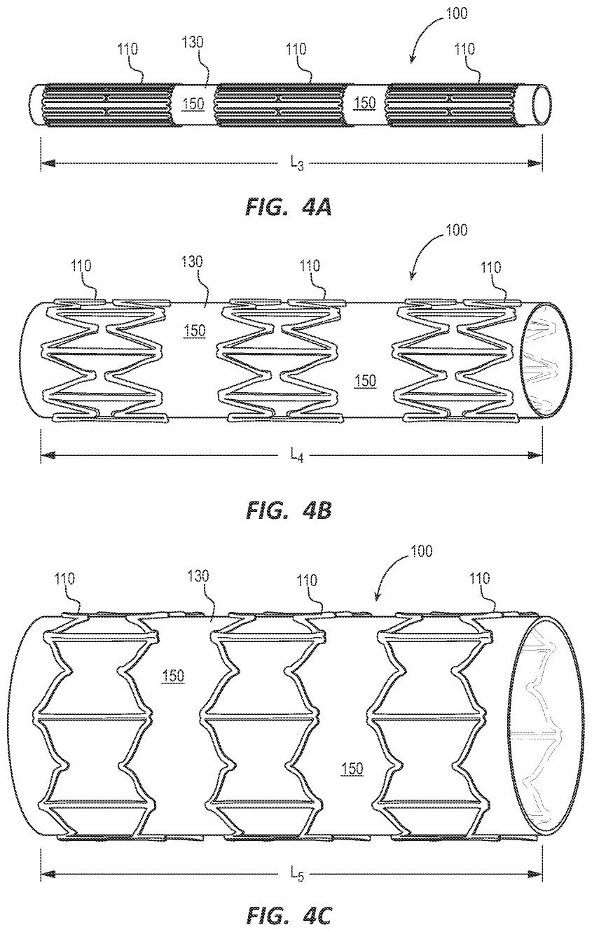

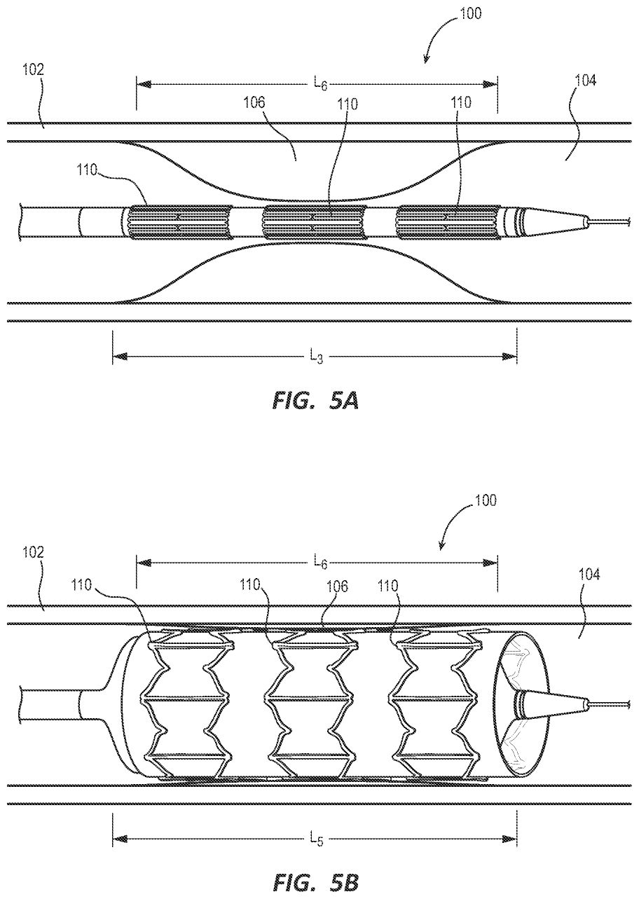

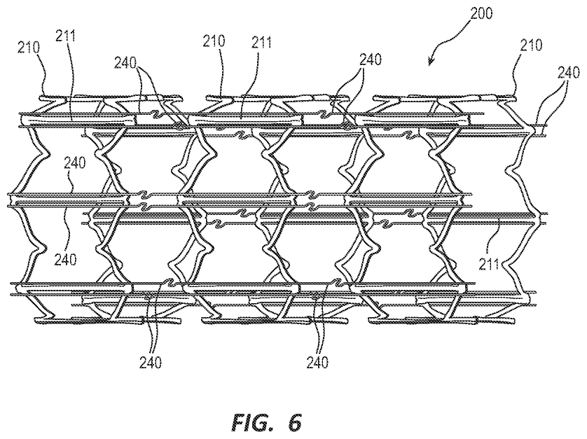

FIG. 1 illustrates an embodiment of a stent device. FIG. 2 illustrates an embodiment of a stent frame of the stent device. FIGS. 3A and 3B illustrate the stent frame in a radially compressed state and expanded state, respectively. FIGS. 4A-4C illustrate the stent device in a radially compressed state, a partial radially expanded state, and an expanded state, respectively. FIGS. 5A and 5B illustrate the stent device within a vessel adjacent a treatment site in the radially compressed state and the radially expanded state, respectively. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

As illustrated in FIG. 1, an embodiment of a stent device 100 includes two broad groups of components; each group may have numerous subcomponents and parts. The two broad component groups are: a stent ring 110 and a coupling member 130. In the depicted embodiment of FIG. 1, the stent device 100 has three discrete stent rings 110. In the illustrated embodiment, each of the discrete stent rings 110 are identical and are positioned equidistance along a length of coupling member 130. In other embodiments, the stent device 100 may include one, two, four, five, or more stent rings 110. In the illustrated embodiment shown in FIG. 1, the stent rings 110 are rotationally aligned to each other forming a uniform array along the length of coupling member 130. In another embodiment, the stent rings 110 may be aligned with a degree of rotation such that a longitudinal strut 111 (See FIG. 2.) of one stent ring 110 may be rotationally offset from the longitudinal strut 11 of an adjacent stent ring 110 thereby creating a sequential pattern of rings that are rotationally misaligned.

The stent rings 110 are disposed along a length of the stent device 100 such that a gap 150 is disposed between adjacent stent rings 110. A length of the gap 150 can range from about zero millimeter to about 10 millimeters and from about 0.5 millimeter to about two millimeters. In some embodiments, the length of the gap 150 can be the same between all of the stent rings 110. In other embodiments, the length of the gap 150 can vary along the length of the stent device 100. For example, the length of the gap 150 may be greater between a stent ring 110 disposed at a distal end of the stent device 100 and an adjacent stent ring 110 than the length of the gap 150 between the stent ring 110 disposed at a proximal end of the stent device 100 and an adjacent stent ring 110. In some embodiments, the length of the gap 150 can vary dependent upon a diameter of the stent rings 110. The gap 150 can allow the stent device 100 to flex between the stent rings 110 such that the stent device 100 can be passed through tortuous body lumens without kinking. In other embodiments, a length of the stent rings 110 may vary from one stent ring 110 to another stent ring 110. Such an embodiment would allow the force required to expand the stent rings 110 to vary along the length of stent device 100 and may allow the stent device 100 to be expanded uniformly by a balloon such that stent rings 110 disposed at a middle portion of the stent device 100 to be expanded at the same approximate rate and manner as the stent rings 100 disposed toward the ends of the stent device 100.

In the illustrated embodiment of FIG. 1, the coupling member is a tube comprising a polymeric material. The polymeric material may be any suitable flexible, biocompatible material. In a particular embodiment, the polymeric material may be polytetrafluoroethylene (PTFE). The polymeric material can include rotationally spun serially deposited PTFE fibers or expanded PTFE (ePTFE). Other long term, biocompatible polymeric materials such as polyester fiber, silicone, urethane, and fluorinated ethylene propylene are contemplated. As also described below, embodiments wherein the coupling member comprises a plurality of filaments rather than a tube of material are likewise within the scope of this disclosure. Furthermore, coupling members wherein the stent rings 110 are coupled by ribbons, sheets, partial tubes filaments, weaves, and so forth are all within the scope of this disclosure.

FIG. 2 illustrates a stent ring 110. As illustrated in the embodiment of FIG. 2, the stent ring 110 includes expandable structures 118. The stent ring 110 can be formed from any suitable material. For example, the stent ring 110 can be formed from a memory alloy, stainless steel, cobalt-chrome, or titanium. The memory alloy can comprise nickel and titanium, such as Nitonol alloys. Other materials are contemplated within the scope of this disclosure. The stent ring 110 may be formed using any suitable technique. For example, the stent ring 110 of FIG. 2 is formed by laser cutting a tubular blank. In other embodiments, the stent ring 110 may be formed by chemically etching a shape using a masking material; stamping or die cutting and wrapping the cut shape around a mandrel and welding; or sintering of a powdered metal using a laser.

In the depicted embodiment, the stent ring 110 includes eight expandable structures 118 coupled together to form a ring shape. In other embodiments, the number of expandable structures 118 may be three, four, five, six, seven, or more. The expandable structure 118 includes a longitudinally oriented strut 111 having a first end 114 and a second end 117 opposite of the first end 114. A length of the longitudinal strut 111 can range from about one millimeter to about 250 millimeters. A diameter of the stent ring 110 in the radially compressed state may range from about one millimeters to about eight millimeters. The diameter of the stent ring 110 in the radially expanded state may range from about two millimeters to about 55 millimeters.

A first strut arm 112 is coupled to the first end 114 and is extendable away from the longitudinal strut 111 in a first direction. A second strut arm 113 is coupled to the first end 114 is extendable away from the longitudinal strut 111 in a second direction opposite of the first direction. A third strut arm 119 is coupled to the second end 117 and is extendable away from the longitudinal strut 111 in the first direction. A fourth strut arm 120 is coupled to the second end 117 and is extendable away from the longitudinal strut in the second direction. The first strut arm 112 is coupled to the second strut arm 113 of an adjacent expandable structure 118. The third strut arm 119 is coupled to the fourth strut arm 120 of the adjacent expandable structure 118. An omega shaped joint 115 couples the first strut arm 112 to the adjacent second strut arm 113 and the third strut arm 119 to the adjacent fourth strut arm 120. A length of the strut arms 112, 113, 119, 120 can range from about 25% to about 50% of the length of the longitudinal strut 111.

In some embodiments the stent ring 110 can be coupled to the coupling member 130 at the longitudinal strut 111 using any suitable technique, such as suture, gluing, bonding, welding, etc. In another embodiment, the stent ring 110 may be coupled to the coupling member 130 at one or more of the strut arms 112, 113, 119, 120 using similar techniques. In other embodiments, the stent ring 110 can be coupled to the coupling member 130 at both the longitudinal strut 111 and one or more of the strut arms 112, 113, 119, 120. In yet another embodiment, the coupling member 130 may include two or more layers of material and the stent ring 110 may be encapsulated by or disposed between the two or more layers.

In certain embodiments, the first end 114 and/or the second end 117 of the longitudinal strut 111 may be bent radially outward relative to a central portion when the stent ring 110 is radially expanded. For example, the longitudinal strut 111 can be bent by a shaped dilatation balloon having features configured to bend the ends 114, 117 radially outward. When bent radially outward, the ends 114, 117 may engage with a vessel wall to prevent axial migration of the stent ring 110.

As shown in FIG. 3A, when the expandable structure 118 is in the radially compressed state, the strut arms 111, 112, 119, 120 are disposed substantially parallel to the longitudinal strut 111. The expandable structure 118 has a length L₁ when the expandable structure 118 is in the radially compressed state. The length L₁ can be substantially equivalent to the length of the longitudinal strut 111. When the expandable structure 118 is in the expanded state, as shown in FIG. 3B, the strut arms 112, 113, 119, 120 may extend away from the longitudinal strut 111 at an angle α ranging from about zero degrees to about 90 degrees. The strut arms 112, 113 are oriented toward the second end 117 of the longitudinal strut 111, and the strut arms 119, 120 are oriented toward the first end 114 of the longitudinal strut 111. The expandable structure 118 has a length L₂ when in the expanded state. The length L₂ can be substantially equivalent to the length of the longitudinal strut 111 and to the length L₁. In other words, the expandable structure 118 is non-foreshortening when transitioning from the radially compressed state to the expanded state and any state in-between. This is accomplished because the longitudinal strut 111 has a fixed or non-changing length that prevents the lengths L₁, L₂ from changing as the expandable structure 118 transitions from the compressed state to the expanded state. The joint 115 is configured to flex open as the strut arms 112, 113, 119, 120 extend away from the longitudinal strut 111 and the angle α increases.

FIG. 4A illustrates the stent device 100 in the radially compressed state having a length L₃. FIG. 4B illustrates the stent device 100 in a partial radially expanded state having a length L₄. FIG. 4C illustrates the stent device 100 in the expanded state having a length L₅. The length L₃ is substantially equivalent to lengths L₄ and L₅. In other words, the stent device 100, comprising the stent rings 110, is non-foreshortening as the stent device 100 transitions from the radially compressed state to the expanded state. Additionally, the length of the gaps 150 (e.g., distance between the stent rings 110) is unchanged as the stent device 100 transitions from the radially compressed state to the radially expanded state.

FIG. 5A illustrates the stent device 100 disposed within a lumen 104 of a vessel 102 adjacent a lesion 106 of a treatment site. As shown, the stent device 100 is in the radially compressed state and has the length L₃. The lesion 106 has a length L₆ and the length L₃ is greater than length L₆. The stent device 100 is positioned such that ends of the stent device 100 extend beyond the borders of the lesion 106. FIG. 5B illustrates the stent device 100 disposed within the lumen 104 of the vessel 102 adjacent the lesion 106 of the treatment site. As shown, the stent device 100 is in the radially expanded state such that the lumen 104 through the lesion 106 is radially opened as the stent device 100 radially outward compresses the lesion 106. The length L₅ of the radially expanded stent device 100 is substantially equivalent to the length L₃ and is greater than the lesion length L₆ such that ends of the stent device 100 extend beyond the borders of the lesion 106 and the lesion 106 is treated along the entire desired treatment length.

It can be appreciated that altering a thickness, width, or material of any of the struts of the stent ring 110 could be done while still maintaining an appearance of similarity of one stent ring 110 to another of the stent device 100, but which would afford varying rates of outward force to be applied against the lesion 106.

FIG. 6 illustrates another embodiment of a stent device 200. As illustrated, the stent device 200 includes a plurality of stent rings 210 disposed along a coupling member 240. The stent rings 210 are substantially similar in structure and function to the stent rings 110 described previously. The coupling member 240 comprises a plurality of elongate filaments 241. In some embodiments, the filaments 241 are coiled. In other embodiments, the filaments 241 are in a braided structure. The filaments 241 can be formed of any suitable flexible, biocompatible material. For example, the filaments 241 can comprise poly-paraphenylene terephthalamide or similar materials. The stent rings 210 can be coupled to the filaments 241 at the longitudinal struts 211 of the stent ring 210.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of manufacturing a stent may include one or more of the following steps: laser cutting a plurality of discrete stent rings from a tubular blank, wherein each discrete stent ring comprises a plurality of expandable structures and wherein each expandable structure comprises a longitudinal strut; disposing the plurality of discrete stent rings along a length of a coupling member; and coupling the longitudinal struts to the coupling member. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use.

References to approximations are made throughout this specification, such as by use of the terms “substantially” or “about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially equivalent” is recited with respect to a feature, it is understood that in further embodiments, the feature can have a precisely equivalent configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A stent frame, comprising:
a plurality of expandable structures, wherein each expandable structure comprises one or more longitudinal struts,
wherein the plurality of expandable structures are coupled together to form a stent ring,
wherein the stent ring comprises a first longitudinal length when in a radially compressed state, and
wherein the stent ring comprises a second longitudinal length equivalent to the first longitudinal length when in a radially expanded state.

2. The stent frame of claim 1, wherein the first and second longitudinal lengths are equivalent to a length of the longitudinal strut.

3. The stent frame of claim 1, wherein each of the plurality of expandable structures comprises:
the longitudinal strut comprising a first end and a second end;
a first strut arm coupled to the first end of the longitudinal strut and extendable in a first direction relative to the longitudinal strut;
a second strut arm coupled to the first end of the longitudinal strut and extendable in a second direction opposite of the first direction relative to the longitudinal strut;
a third strut arm coupled to the second end of the longitudinal strut and extendable in the first direction relative to the longitudinal strut; and
a fourth strut arm coupled to the second end of the longitudinal strut and extendable in the second direction relative to the longitudinal strut.

4. The stent frame of claim 3,
wherein the first strut arm is coupled to the second strut arm of a first adjacent expandable structure,
wherein the second strut arm is coupled to the first strut arm of a second adjacent expandable structure,
wherein the third strut arm is coupled to the fourth strut arm of the first adjacent expandable structure, and
wherein the fourth strut arm is coupled to the third strut arm of the second adjacent expandable structure.

5. The stent frame of claim 3, wherein when the ring is in the radially expanded state,
the first strut arm is disposed at angle ranging from zero degrees to 90 degrees relative to the longitudinal strut,
the second strut arm is disposed at angle ranging from zero degrees to 90 degrees relative to the longitudinal strut,
the third strut arm is disposed at angle ranging from zero degrees to 90 degrees relative to the longitudinal strut, and
the fourth strut arm is disposed at angle ranging from zero degrees to 90 degrees relative to the longitudinal strut.

6. The stent frame of claim 4,
wherein a first flexible joint is disposed between the first strut arm and the second strut arm of the first adjacent expandable structure,
wherein a second flexible joint is disposed between the second strut arm and the first strut arm of the second adjacent expandable structure,
wherein a third flexible joint is disposed between the third strut arm and the fourth strut arm of the first adjacent expandable structure, and
wherein a fourth flexible joint is disposed between the fourth strut arm and the third strut arm of the second adjacent expandable structure.

7. The stent frame of claim 6, wherein the first, second, third, and fourth flexible joints comprise an omega shape.

8. The stent frame of claim 1, wherein the plurality of expandable structures comprise any one of a shape memory metal alloy, stainless steel, cobalt-chrome, or titanium.

9. The stent frame of claim 3, wherein one or more of the first end and the second end of the longitudinal strut is bent radially outward relative to a central portion of the longitudinal strut.

10. The stent frame of claim 1, wherein each of the plurality of expandable structures is laser cut from a tubular blank.

11. A stent, comprising:
a plurality of discrete stent rings comprising a plurality of expandable structures coupled together, wherein each expandable structure comprises one or more longitudinal struts; and
a coupling member configured to operably couple the plurality of discrete rings.

12. The stent of claim 11,
wherein the coupling member is a polymeric tube,
wherein the plurality of discrete stent rings are disposed along a length of and surround the polymeric tube, and
wherein each of the longitudinal struts is coupled to the polymeric tube.

13. The stent of claim 12, wherein one or more of first, second, third, and fourth strut arms of each of the plurality of expandable structures are coupled to the polymeric tube.

14. The stent of claim 12, wherein the polymeric tube comprises one or more of polytetrafluoroethylene, fluorinated ethylene propylene, and silicone.

15. The stent of claim 11, wherein the coupling member comprises a plurality of elongate filaments,
wherein the plurality of discrete stent rings are disposed along a length of the plurality of elongate filaments, and
wherein the longitudinal struts are coupled to the plurality of elongate filaments.

16. The stent of claim 15, wherein the plurality of elongate filaments comprise poly-paraphenylene terephthalamide.

17. The stent of claim 11,
wherein the plurality of discrete stent rings are disposed along a length of the coupling member; and
wherein the one or more longitudinal struts of a first discrete stent ring are rotationally offset relative to the one or more longitudinal struts of a second discrete stent ring.

18. A method of manufacturing a stent, comprising:
laser cutting a plurality of discrete stent rings from a tubular blank, wherein each discrete stent ring comprises a plurality of expandable structures and wherein each expandable structure comprises a longitudinal strut;
disposing the plurality of discrete stent rings along a length of a coupling member; and
coupling the longitudinal struts to the coupling member.

19. The method of claim 18, wherein the coupling member is a polymeric tube.

20. The method of claim 19, wherein the coupling member comprises a plurality of filaments.
